(19) United States
(12) Patent Application Publication
(10) Pub. No.: US 2004/0067599 A1
(43) Pub. Date: Apr. 8, 2004

(54) Separation identification and quantitation of protein mixtures

(76) Inventors: Joseph L. Katz, Baltimore, MD (US); Denis Wirtz, Washington, DC (US)

Correspondence Address: WHITHAM, CURTIS & CHRISTOFFERSON, P.C., 11491 SUNSET HILLS ROAD, SUITE 340, RESTON, VA, 20190, US

(21) Appl. No.: 10/450,194

(22) PCT Filed: Dec. 14, 2001
(86) PCT No.: PCT/US01/47754

(51) Int. Cl. G01N 33/543; C12Q 1/68; C07K 1/00

(52) U.S. Cl. 436/518; 435/6; 530/413

(57) ABSTRACT

A Repeated Classification Procedure (RCP) for the rapid separation, identification, classification, and quantitation of proteins in a mixture of proteins utilizes general-motif antibodies which typically bind to a plurality of proteins in a mixture which have a common motif or are homologs in some respect. By repeatedly using general-motif antibodies or using general-motif antibodies in conjunction with specific antibodies, a pattern of binding can be produced which uniquely identifies proteins of interest. By using immobilized general-motif antibodies to effect separation, the identification, classification, and quantitation of proteins in a sample can be use to characterize the protein synthesis pattern of disease states. Quantitation of protein in a sample can be determined based on the movement of very small beads with bound protein in a magnetic field, wherein larger quantities of bound protein causes the beads to travel relatively smaller distances or at relatively slower rates.

### BACKGROUND OF THE INVENTION

1. Field of the Invention

The invention generally relates to the rapid separation, identification, classification, and quantitation of proteins in a mixture of proteins. In a particular embodiment, the invention provides a method of repeatedly using “separation linkers” to effect the separation, identification, classification, and quantitation of proteins in order to characterize patterns of protein synthesis patterns. The invention further provides general-motif antibodies for use as separation linkers in the methods of the present invention. The invention further provides a means of determining protein concentrations in a sample by measuring their differential migration in a magnetic field of magnetic beads containing bound protein.

### BACKGROUND OF THE INVENTION

Healthy tissues typically differ from diseased tissues in their levels of protein expression. Development of a disease state may be characterized by:

1) different patterns of protein expression, e.g., a subset of proteins may be upregulated or downregulated;

2) the generation of new proteins, e.g. the actin-binding protein fascin, which is undetectable in naive B cells, becomes expressed upon infection of B cells by the Epstein-Barr virus; and

3) interactions among proteins may be initiated or enhanced, e.g. a subset of proteins may become activated and, as a result, trigger or mediate signaling pathways which involve new protein interactions. Levels of calcium and/or the intracellular pH also may change upon viral infection, resulting in new interactions among proteins or the modulation of existing interactions.

Currently, diagnostic procedures for detecting and identifying disease states usually rely on the detection of a single or only a few protein markers. This approach is limited in that it does not take into account the fact that most disease states are characterized by a variety of alterations in several proteins. One result of this oversimplified detection methodology is the frequent occurrence of undesirable “false positives” and “false negatives” due to the utilization of a single or only a few markers.

It would be highly advantageous to have available a method for detecting the pattern of alterations in protein expression which is characteristic of a disease state.

Also, currently in order to separate and identify individual proteins from a mixture of proteins, monoclonal or polyclonal antibodies frequently are used. The utility of such antibodies, however, is limited when the mixture includes a large number of proteins (e.g. several hundred or more) and the goal is to identify many or all of the proteins in the mixture, because this necessitates using one antibody for each protein. It would be highly desirable to have available a method for separating and identifying proteins in a mixture of a large number of proteins without having to generate a specific antibody for each protein. Further, it would be especially advantageous to have available a technique which (in addition to allowing the separation and identification of all proteins in a mixture of a large number of proteins) would allow the concomitant quantitation of each protein, and would also provide functional and structural information about each protein.

The ability to separate and quantitate the proteins in samples containing a mixture of proteins is of fundamental importance to many scientific and technological endeavors. Generally, separation and quantitation are carried out separately. One technique that is currently used in the process of separating proteins is the use of antibodies to sequester the proteins for which they are specific. For example, antibodies may be immobilized on a substrate and exposed to a mixture of proteins. Those proteins which bind the antibodies become tethered to the substrate via the antibody and may then be removed from the mixture by removal of the substrate. One example of this is the use of antibodies which are attached to magnetic beads. However, while this technique is very useful for separating proteins, quantitation of the amount of bound protein may involve further extensive manipulations. It would be highly desirable to have available a method which allows the direct quantitation of antibody-bound protein.

### SUMMARY OF THE INVENTION

The invention provides a method of separating proteins that are present in a mixture of proteins by utilizing a set of binding agents, hereafter denominated “separation linkers” or “slinkers”. Separation is accomplished by exposing the mixture of proteins to the separation linkers (either concomitantly in, for example, an array of slinkers, or sequentially, i.e. one separation linker at a time), and then separating the proteins that bind to each linker from those that do not. The bound proteins may be released from the linkers if desired. The slinkers may be immobilized directly onto a substrate (for example, magnetic beads, microslides, rods, fibers, chromatographic columns, and the like) and the immobilization may be via a spacer molecule or a secondary antibody. The slinker may be an antibody (specific or general) or other types of linker molecules. The proteins that are separated may interact with each other or may be non-interactive. The proteins and the slinker may be labeled with a detectable label, for example a fluorescent, colorimetric or chemiluminescent label. The method also may include separating proteins using a slinker in which the affinity of a protein for the slinker is adjusted by changing the environment of the binding reaction, e.g. by adjusting the temperature, pH, ionic strength or composition of buffer, and the like. The method further may be coupled with the use of other protein separation techniques, e.g. various forms of chromatography or electrophoresis.

The present invention also provides a method for quantifying the amount of protein in a sample. In the method, the sample is contacted with a magnetic bead that is coated with a slinker which binds the protein. The speed or the distance of migration in a magnetic field of the magnetic bead with attached protein is measured and correlated to the quantity of protein that is attached. Further differentiation of migrating beads may be effected by subjecting the beads to a transverse field (e.g. electric, flow, or magnetic) prior to, simultaneously with, or after placing the beads in the main magnetic field. Further, different slinkers may be attached to beads of different sizes, providing an additional differentiation mechanism. Also provided is an apparatus for measuring protein content in a sample. The apparatus includes a magnetic bead, slinkers associated with the bead, a means for monitoring the rate of migration of the bead in a magnetic field, and a means to calculate the quantity of protein bound to the bead based on the measured speed of migration. The apparatus may further include a substrate with microchannels, the cross sections of which are larger than the diameter of the magnetic beads, and sensors to sense the movement of the magnetic beads. The slinkers employed may be antibodies (general-motif or specific). However, other types of slinkers may be used in the practice of this invention (e.g. actin, tubulin, integrins, substances that bind phosphorylated groups, etc.). The magnetic beads may preferably be nanobeads having a radius in the range of about 5 to 1000 nm; however, other materials and size ranges may be suitable for use in the practice of the present invention.

The invention further provides a classification database for proteins based on their binding signatures. The binding signatures reflect the pattern of binding/not binding to the set of slinkers utilized in the methods of the invention. i.e. the Repeated Classification Procedure data. To that end, the invention also provides a method of generating such a protein classification database. The method involves exposing each protein to be classified to a set of slinkers, detecting which of said slinkers bind to each protein, and compiling a database from this information. Those of skill in the art will recognize that such a database can be utilized as a way of cataloging information for normal and disease states. Such a database then would aid in the diagnosis of diseases by allowing the comparison of binding signatures from unknown samples to those of normal and of known disease states. If a binding signature differed from the normal binding signature, that could suggest the presence of a disease state, especially if the signature matched that of a known disease state. Such information could be used in concert with other diagnostic techniques for confirmation, many of which are well-known to those of skill in the art.

The present invention also provides a method for quantitating the proteins present in a mixture of proteins using optical fibers. In this method, the protein mixture is labeled with a non-specific fluorescent dye. A bundle of optical fibers is then immersed in the protein mixture. Each fiber in the bundle having previously been coated with a different slinker. The bundle is removed from the mixture and light is shined on each fiber in turn, inducing fluorescence in those fibers which have fluorescently labeled proteins attached. The intensity of fluorescence is correlated with an amount of protein.

The invention also provides a method for generating general-motif antibodies (a subset of slinkers). The method involves identifying a protein motif that is common to all the proteins in a given set of proteins and which is antigenic, and producing an antibody to this motif. Examples of such motifs include regions of primary, secondary, or tertiary structural homology in the proteins.

### BRIEF DESCRIPTION OF THE DRAWINGS

FIG. 1. A schematic representation of the use of RCP to separate and categorize eight non-interacting proteins (a, b, c, d, e, f, g, and h) using three different slinkers (α, β, and γ). 0 is used to denote the subsets of proteins which were not bound by a slinker.

FIG. 2. A schematic representation of the use of RCP to separate four proteins, a, b, c, and d, two of which (a and c) bind strongly to each other at the solution conditions being used, using slinkers α and β. 0 is used to denote the subsets of proteins which were not bound by that slinker. The asterisk is used to denote the amount of protein c which was not bound to protein a in the original mixture.

FIG. 3. Example of a two-level separation in which ten proteins are separated using five different slinkers. Each of the ten proteins binds strongly to two of the slinkers.

FIG. 4. Example of a two-level separation in which ten proteins are separated using five different slinkers. This shows that it is possible to separate the ten proteins even when each binds weakly to two of the slinkers. Thus, not all of that protein is removed in a single step. Remaining protein is indicated by a “bar” (-) over the protein name.

FIG. 5. Example of a three-level separation in which ten proteins are separated using five different slinkers. Each of the ten proteins binds weakly to three of the slinkers.

FIG. 6. Example of a three-level separation in which twenty proteins are separated using six different slinkers. Each of the twenty proteins binds weakly to three of the slinkers.

FIG. 7. Schematic depiction of a microarray for use in RCP.

FIG. 8. Schematic depiction of a device for quantitating the amount of protein bound to magnetic beads.

FIG. 9. Schematic depiction of a portion of the device shown in FIG. 4 which includes a photographic device used to evaluate bead movement.

FIG. 10. Illustration of conversion of a grey-scale image of a packet in a channel to a thresholded black and white image.

### DETAILED DESCRIPTION OF THE PREFERRED EMBODIMENTS OF THE INVENTION

### Repeat Classification Procedure

The present invention provides a procedure, denominated the Repeated Classification Procedure (RCP), to separate, identify, and classify macromolecules obtained from a biological sample rapidly and quantitatively. In preferred embodiments of the present invention, RCP allows the detection of the presence of new proteins, new or enhanced protein interactions, and/or different concentrations of proteins in a single procedure. In other embodiments, the macromolecules which are detected may be nucleic acid molecules such as mRNA or DNA, or any ohter type of large molecules (glycoproteins, starches, fats, etc.) which can be attached to an appropriate slinker. RCP, therefore, offers a robust test of a change in state (for example, from a normal to a disease state) by detecting a combination of differences between states that may result from many causes.

Utilization of RCP provides a means to determine, (as desired, part or all of) the pattern of molecular expression for a biological sample. Such biological samples include but are not limited to extracts from the cytoplasm of (both prokaryotic and eukaryotic) cells (e.g. mammalian, plant, fungus, bacterial etc.), or from various subcellular organelles such as the nucleus, mitochondria, the endoplasmic reticulum, chloroplasts, and the like. Further, other types of samples may be assayed as well, including blood, plasma, amniotic fluid, tissue samples, biopsy samples, and the like. Those of skill in the art will recognize that the methods of the present invention may be used to assess the pattern of molecular expression in a wide variety of biological samples, all of which applications are intended to be encompassed by the present invention. For example, by comparing the protein pattern from diseased tissue to that of healthy tissue, it is possible to establish a “pathological signature” for a particular disease state. By “pathological signature” we mean the pattern of molecular (e.g. protein) expression which is characteristic of a particular disease state. This signature is comprised of a combination of markers and thus renders the diagnosis more reliable and more informative than currently used procedures which rely on a single or only a few markers.

An example of a disease state that is amenable to diagnosis by the methods of the present invention is Epstein-Barr virus (EBV) infection. When naive B lymphocytes become infected by Epstein-Barr virus, many proteins change their expression levels. Some of these proteins include the two cytoskeletal proteins fascin and MARCK, as well as the C3d receptor, the 45 kDa-lymphoblastoid associated protein and the C-cell restricted activation antigen. Fascin expression is routinely used as a marker for EBV infection. Fascin, however, also becomes overexpressed in certain cancer cells upon transformation. Hence, the diagnosis of EBV infection would be more accurate if the entire pattern of protein expression was detected rather than a single protein. Alternatively, changes in the expression of a combination of several relevant proteins would also suffice to accurately monitor the disease state.

Basically, RCP involves the step-wise, sequential exposure of a mixture of macromolecules to a set of slinkers. In a preferred embodiment of the present invention, the macromolecules are proteins. In other embodiments of the present invention, the macromolecules are mRNA or DNA. In yet other embodiments, combinations of, for example, proteins and mRNA, or proteins and DNA, may be detected by the methods of the present invention. The slinkers may be immobilized on a solid support or may be used without a support. In a preferred embodiment of the present invention, the slinkers are “general-motif antibodies”, a full description of which is given below. However, any suitable slinker may be utilized in the practice of the present invention.

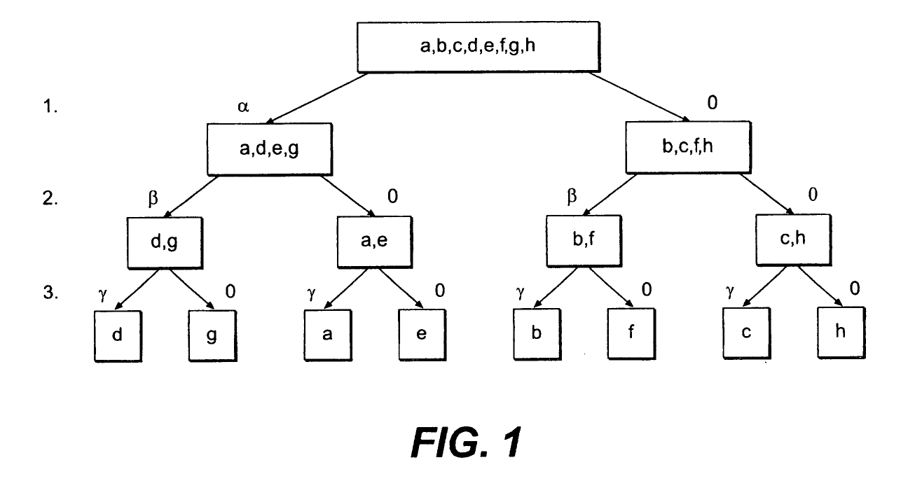

For example, Table 1 summarizes the potential interactions of proteins a, b, c, d, e, f, g, and h with slinkers α, β, and γ. In Table 1, “1” indicates strong binding between the protein and indicated slinker, and “0” indicates no binding. A schematic representation of the use of RCP to separate and categorize these eight proteins (a, b, c, d, e, f, g, and h, which do not interact with each other) using a set of three different slinkers (α, β, and γ) is depicted in FIG. 1. As can be seen, at each step or level of separation, the immobilized slinker used at that level separates the proteins in the mixture into two different protein subsets or protein pools. It is important to note that in this Figure and in other Figures and implementations described throughout, unless otherwise indicated, each reference to a single protein (e.g. “protein d”) actually means “all proteins which satisfy the specified binding conditions”. Thus, “protein d” may represent a single protein or a group of proteins. For example, in FIG. 1, the words “protein d” represent all proteins in the mixture that bind slinkers α and β but not slinker γ. Similarly, “protein h” represents all proteins in the mixture that do not bind slinkers α, β, and γ.

TABLE 1
Interactions between 8 proteins, a, b, c, d, e, f, g,
and h, and 3 slinkers α, β, and γ.

| | α | β | γ |
|---|---|---|---|
| a | 1 | 0 | 0 |
| b | 0 | 1 | 0 |
| c | 0 | 0 | 1 |
| d | 1 | 1 | 0 |
| e | 1 | 0 | 1 |
| f | 0 | 1 | 1 |
| g | 1 | 1 | 1 |
| h | 0 | 0 | 0 |

Process1 of FIG. 1, shows the binding of slinker α to proteins a, d, e, and g. When the immobilized α is removed from the sample, the proteins which are bound to it also are removed, forming a subset pool that contains proteins a, d, e, and g. A second subset pool contains the proteins b, c, f, and h (i.e. those which do not bind α) which were left behind (symbolized by the “0” arrow). In turn, both protein subset pools then are exposed to a second slinker β (i.e., Process 2 in FIG. 1), which divides each of them into two additional pools, yielding a total of four subset pools (d,g; a,e; b,f; and c,h). The proteins in one of the four pools (d,g) bind both the first and second slinkers; the proteins in a second pool (a,e) bind only the first but not the second slinker; the proteins in a third pool (b,f) bind only the second and not the first slinker; and the proteins in the fourth pool (c,h) do not bind either the first or the second slinkers. Each of the four subset pools is then exposed to a third slinker γ (i.e., Process 3 of FIG. 1), and so on, until the desired degree of separation is achieved, i.e. until each subset pool created by exposure to the various slinkers being applied contains only a single protein or only a desired subset of proteins. The separation may be complete (i.e. the final protein pools each contain a single protein), the separation may be partial (the final protein pools contain more than one protein), or some final pools may contain only one protein while others contain more than one protein, depending on the purpose of a particular investigation.

Further, those of skill in the art will recognize that in each process, one can use different slinkers for some subpools than for others. For example, after application of slinker α, two or more different slinkers (e.g. β1 and β2) can be applied instead of a single slinker β. Likewise, at the next level of separation, two or more γ slinkers may be applied.

RCP may be applied to mixtures of proteins in which the proteins do not interact with each other (e.g. FIG. 1), and also to mixtures of proteins in which some of the proteins do interact with each other. In the latter case, RCP can provide valuable information about the strength of the interaction and the relative concentrations of the interacting proteins. Table 2 summarizes all possible interactions between the four proteins a, b, c, and d and two slinkers α and, β, where “1” indicates strong binding and “0” indicates negligible or no binding.

TABLE 2
Possible interactions between 4 proteins, a, b,
c and d, and 2 slinkers α and β.

| | α | β |
|---|---|---|
| a | 1 | 0 |
| b | 0 | 1 |
| c | 0 | 0 |
| d | 1 | 1 |

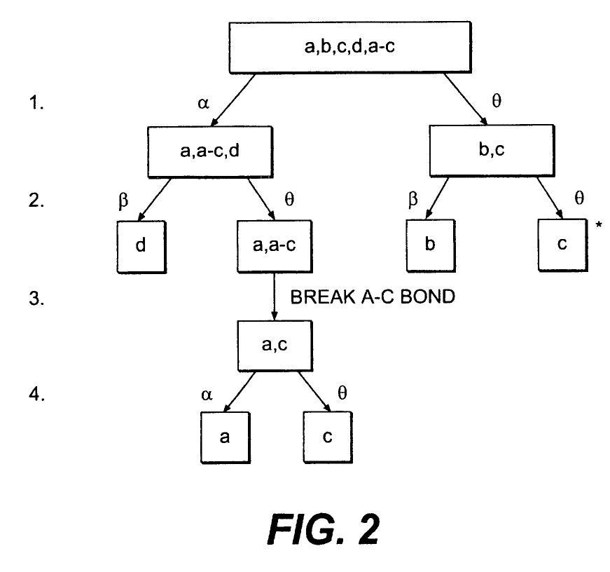

In the case of interacting proteins, the pattern of binding will differ from that of non-interacting proteins. A strategy for separating four proteins, a, b, c, and d, two of which (a and c) bind strongly at the solution conditions being used, using slinkers α and β is depicted is FIG. 2. In Process 1 of FIG. 2, a mixture of proteins a, b, c, d and a-c complexes are exposed to a first slinker α. Slinker α, together with its bound proteins, is then removed thus dividing the sample into two protein pools. One pool contains the proteins which bind to α (a, d, and a-c complexes); the other pool contains proteins b and the molecules of c which were not bound to a. In Process 2 of FIG. 2, a second slinker, β, which binds proteins b and d, is used to separate protein d from the first pool and protein b from the second pool. At this stage of the method, proteins d, b, and c are separated, except for those molecules of protein c that are bound in a-c complexes and thus also are present in the pool containing a. Proteins a and c then are separated from each other by breaking the a-c bond (Step 3. e.g. denaturing by a change in pH or ionic strength) and re-exposing the resulting a, c protein pool to slinker α (Step 4). Note that when conditions are such that a and c form an extremely strong bond, one obtains the protein c pool (marked with an asterisk) only when c is in excess in the original mixture. When the a-c bond is not extremely strong, there always will be some protein c which will be left behind in the α separation step and thus will need to be separated in the β separation step. Note also that the ratio of the amounts of c obtained via the two different separation paths provides information about the ratio of concentration of a versus c whenever the a-c bond is strong, and otherwise (when the ratio of initial concentrations is known) information about the strength of the a-c interaction.

A modification of RCP can be used to identify the proteins in a cell or tissue extract which interact with each other. For example, a mixture containing the protein a and three proteins b, c, and d which interact strongly with a may be analyzed in the following manner: the cell extract is mixed with immobilized α, a slinker with affinity for a. The protein pairs a-b, a-c, and a-d then are separated from the cell extract by removing this slinker with its attached proteins; all proteins that do not interact with a remain in the cell extract. The immobilized α-a complexes are then exposed to slinkers which are highly specific for b, c, and d, and which themselves are differentially labeled, for example, fluorescently labeled antibodies. When the resulting complexes are examined, it is possible to identify and quantify the concentrations of the a-b, a-c, and a-d pairs.

A modification of RCP can be used to quantify the affinity of a protein for other proteins. In a mixture containing two interacting proteins, a and b, if a and b have a strong affinity, three situations are possible: 1) [a]=[b]; 2) [a]<[b]; and 3) [a]>[b]. (Braces are used to indicate concentration.) In the first situation, exposing the sample to an α slinker (i.e. specific for a) will remove essentially all of a and all of b as a-b pairs. In the second situation, a-b pairs are removed but some proteins of type b are left behind. In either of these cases, a slinker β which is specific for b then is used to remove the remaining b protein. In the third situation, free a is removed as well as all of the b, as a-b pairs. Proteins a and b are subsequently separated by, for example, changing the pH and/or ionic strength and using slinker β.

In some embodiments of the present invention, the slinkers that are utilized in the RCP procedure are antibodies. The antibodies may be specific antibodies, the production of which is well-known to those of skill in the art, or general-motif antibodies. The production of general-motif antibodies is described in detail below. However, those of skill in the art will recognize that other types of slinkers may be utilized, either alone, or in combination with antibodies to carry out RCP. For example, any molecule which contains a binding site for a suitable fraction of the molecule(s) of interest (e.g. proteins in a protein mixture) may be utilized as slinkers in the practice of the present invention. Examples of molecules which may function as slinkers are specific antibodies, enzyme substrates and substrate analogs, co-factors, coenzymes, inhibitors, general-motif antibodies, protein-binding DNA and RNA sequences, nucleic acids, metal ions, saccharides, spacer molecules with accessible functional groups (e.g. amino or sulfhydryl functions), integrins, selecting, cadherins, intermediate filaments, vimentin, neurofilaments, keratins, components of receptor-ligand pairs, and the like. Slinkers also may be full length proteins and peptides. Some examples are cytoskeleton filaments such as actin and microtubule (a polymer of tubulin) which can bind many cytoskeleton proteins. These proteins can be used, for example, to identify proteins that bind both microtubule and actin. By “spacer molecule” we mean molecules typically utilized by those of skill in the art to provide additional distance between the surface of a substrate and an active moiety which is tethered to the substrate, but which is itself relatively inactive, for example an alkyl chain. Any molecule which is capable of binding a suitable fraction of the molecules in a sample of interest may be used in the practice of the present invention. Any combination of slinkers may be used to form a set of slinkers for use in the practice of the present invention, so long as the use of the set of slinkers results in a satisfactory separation and identification of the proteins in the sample of interest.

The suitability of a slinker for inclusion in a particular set of slinkers, and the precise order in which the slinkers are used, is predicated on the results of experimental screening. Basically, potential slinkers are tested on a variety of appropriate samples. For example, protein samples may include: original samples of interest (e.g. tissue samples); subsequent pools of protein which result from an exposure of a sample to a potential slinker; samples of purified protein; mixtures of purified proteins, and the like. Those of skill in the art will recognize that many different protein mixtures would be appropriate for testing the activity of potential slinkers, and for optimizing the order of a sequence of slinkers. The data which is obtained in this fashion is utilized to generate sets of slinkers, which may be used in the practice of the present invention. Further, the order in which potential slinkers are exposed to samples also should be varied in order to test and optimize the efficacy of the sets of slinkers with respect to a sample of interest.

The sets of slinkers so developed may be utilized sequentially (i.e. in a given order), simultaneously in arrays, or a few at a time for a given application.

The nature of the slinkers to be used may be coordinated with and based on genomic information. Using the information obtained by initial genomic screening (e.g. by the use of gene microarrays), microarrays or custom sets of slinkers can be designed which contain only slinkers corresponding to the macromoleucles (e.g. proteins or mRNA) produced under a given set of circumstances, e.g. in a specific tissue type, or during a particular disease process.

A major advantage of RCP is its dramatic reduction in the number of binding agents (relative to the use of, for example, specific antibodies) needed to accomplish a separation. In one embodiment of the present invention, each slinker used in a separation scheme binds about half of the proteins in a protein sample. In this case, the minimum number of slinkers, n, required to identify N proteins using RCP is (log N/log 2). Thus, if there are 100,000 different proteins in the initial mixture, then in principle as few as 17 different slinkers would suffice to completely separate them using RCP. In contrast, 99,999 specific antibodies would be required to effect the same separation. Further, the use of specific antibodies requires prior knowledge of the identity of all proteins in the mixture so that the appropriate antibodies can be obtained or produced.

However, the above-described case (as well as the separation schemes illustrated in FIGS. 1 and 2) are “ideal” situations in which a slinker binds about half of the proteins in a sample. As more usually will be the case, in other embodiments of the present invention each slinker binds much more or much less than half of the proteins in the mixture, therefore, more slinkers will be required to effect the desired level of separation. Additionally, more slinkers will be required if there is information degeneracy within the protein sample, which is likely to be the case. For example, in the three-slinker case illustrated in FIG. 1, slinker α binds to proteins a, d, e, and g but it does not bind to proteins b, c, f, and h; similarly, slinker β binds to b, d, f, and g but not to a, c, e, and h. If any of these conditions is not satisfied the procedure still will work, but it will require the use of at least one additional slinker to make a complete separation. For example, if β does not bind f, then after Process 2 one is left with a protein group consisting of c, h, and f instead of only c and h. Process 3 (i.e. the use of γ) would be inadequate to fully separate three proteins. Assuming that γ binds to neither f nor h, step 3 separates c from f and h and an additional step (i.e., the use of δ) would be required to separate f from h. Furthermore, if one does not know whether f and h each represents only one protein, those of skill in the art will recognize that additional steps may be required and incorporated into the RCP protocol.

RCP is not limited to separations involving a large number of successive steps or processes of separation. RCP also works by successively applying slinkers to a given pool of proteins using only a small number of processes of separation. Examples of two- and three-process procedures are described below. Those of skill in the art will recognize that many multi-process combinations of procedures can be designed for use according to the methods of the present invention, and all such combinations are intended to be included in the scope of the present invention.

Separation of Two-Slinker Proteins: Consider a mixture consisting of ten different proteins, a-j, each of which binds to two (and only two) of five slinkers α, β, γ, δ, and ε (see Table 3 for the list of potential interactions; 1 represents strong binding, 0 represents negligible binding). These ten proteins can be fully separated by applying general-motif antibodies α, β, γ, δ, and ε. At each step of the separation procedure of this particular illustration, the use of sufficient slinker is assumed to remove essentially all of the desired protein (e.g., more than 99% or 99.9% or whatever purity is desired).

TABLE 3
Binding Pattern for Ten Proteins that each Bind to Two Slinkers

| | Slinkers | | | | |
|---|---|---|---|---|---|
| Proteins | α | β | γ | δ | ε |
| a | 1 | 1 | 0 | 0 | 0 |
| b | 0 | 1 | 1 | 0 | 0 |
| c | 0 | 0 | 1 | 1 | 0 |
| d | 0 | 0 | 0 | 1 | 1 |
| e | 1 | 0 | 0 | 0 | 1 |
| f | 1 | 0 | 1 | 0 | 0 |
| g | 0 | 1 | 0 | 1 | 0 |
| h | 0 | 0 | 1 | 0 | 1 |
| i | 1 | 0 | 0 | 1 | 0 |
| j | 0 | 1 | 0 | 0 | 1 |

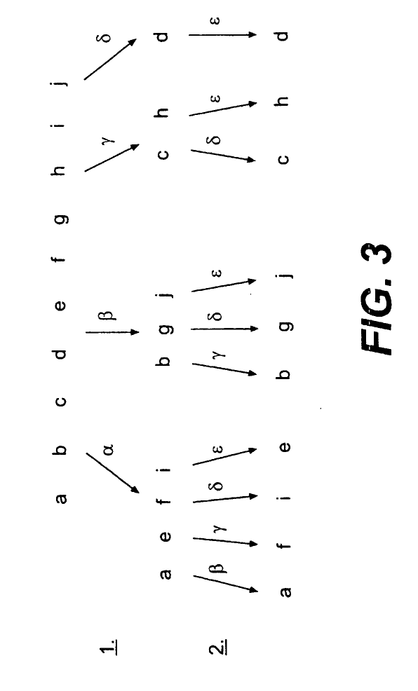

As illustrated in FIG. 3, applying slinker α to the protein pool removes a, e, f and i from the pool. Then applying β to the remaining proteins in the pool (i.e., at the same level of processes) removes b, g, and j. (Note that in this and related figures, the order of the successive applications of different slinkers in a process should coincide with the order from left to right as depicted in the figure.) Slinker γ then removes c and h, and δ removes d. Note that if one were certain that there were only these ten proteins in that mixture, the δ removal is not necessary. However using δ allows this also to be the procedure for removing ten particular proteins from a mixture of ten or more proteins.

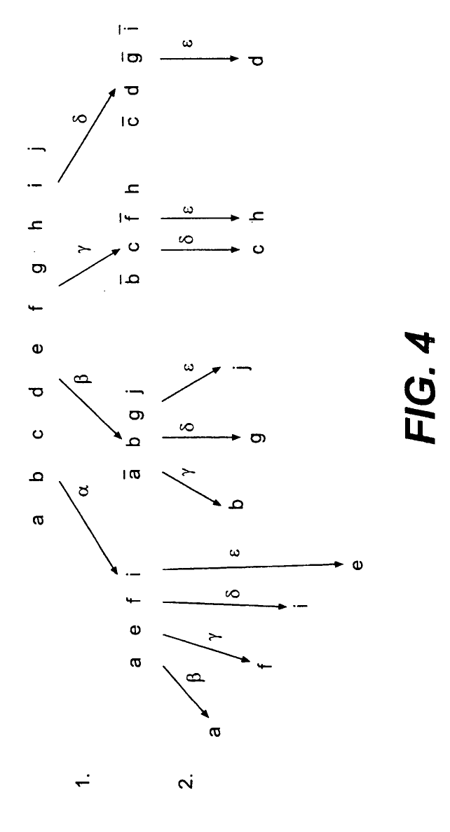

In the second process of this separation procedure, slinkers β, γ, δ, and ε, are successively applied, thus separating each group created in the first process into the individual proteins. (Note that the last separation shown, using ε on the group which only has d in it is unnecessary; it is included here to facilitate the explanation of FIG. 4 in the next paragraph.)

Sometimes it may be useful or necessary to utilize a slinker whose affinity for some proteins is sufficiently weak that, at the concentration of slinker which is practical or convenient to add to the protein mixture, most but not all molecules of those proteins are extracted from the mixture. Nonetheless, one still can apply RCP to the proteins in the mixture. This is illustrated by FIG. 4 which shows a separation identical to that shown in FIG. 3, except that some protein is “left behind” by each of the slinkers utilized in the first process. In this illustration, the protein that is left behind is entirely removed on exposure to the next slinker which binds to it. The protein left behind after the first application of a general antibody is indicated by a bar over the roman letter which identifies it, e.g., \overline{a}. As can be seen in FIG. 4, complete separation can be effected by this scheme. Thus, even when the slinkers to be applied are not ideal, i.e. when not all molecules of protein a bind to slinker α, RCP remains applicable and useful.

The number of slinkers required to separate a given number of proteins when each protein binds to more than one slinker can be calculated. Assuming, as in the above example, that each protein binds to exactly two slinkers, if m equals the number of proteins one desires to separate and n is the number of slinkers, then n is related to m by the equation:

m≦n!/2(n−2)!=n(n−1)/2 Equation 1

Thus, to separate up to 190 proteins 20 slinkers will suffice, and to separate 100,000 proteins, 448 slinkers will suffice, etc. Thus, even though each protein binds to only two of the linkers, one causes a drastic reduction in the number of binding agents which, without RCP, otherwise would be needed.

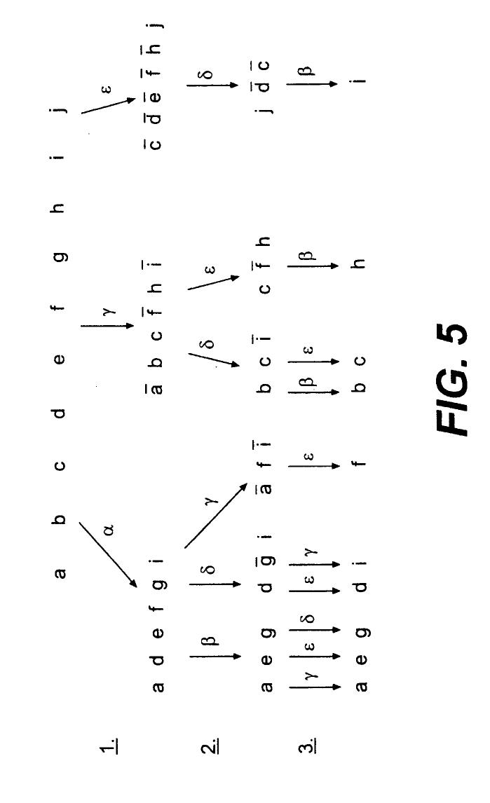

Separation of Three-Slinker Proteins: Those of skill in the art will recognize that, in addition to binding to two-slinkers, other schemes can be implemented in the RCP. For example, consider ten proteins a-j and five slinkers α-ε where each protein binds (not extremely strongly) to three of the slinkers (see Table 4 for the interactions between proteins and slinkers). As illustrated in FIG. 5, in the first process, slinkers α, γ, and ε are successively applied. In process 2, three, two, and one slinker(s), respectively, are successively applied to the three subsets of proteins that were generated in the first level of separation. In the third and (for ten or fewer proteins) last process, all proteins are separated by following the pattern of application of slinkers given in FIG. 5. Note that FIG. 5 assumes that binding to the slinkers is sufficiently weak that some protein is left behind (as in the example illustrated by FIG. 4). When only strong affinity slinkers are used, the procedure is the same except that (since no protein is left behind) the letters with the overbars disappear.

TABLE 4
Binding Pattern for Ten Proteins that each Bind to Three Slinkers

| | Slinkers | | | | |
|---|---|---|---|---|---|
| Proteins | α | β | γ | δ | ε |
| a | 1 | 1 | 1 | 0 | 0 |
| b | 0 | 1 | 1 | 1 | 0 |
| c | 0 | 0 | 1 | 1 | 1 |
| d | 1 | 0 | 0 | 1 | 1 |
| e | 1 | 1 | 0 | 0 | 1 |
| f | 1 | 0 | 1 | 0 | 1 |
| g | 1 | 1 | 0 | 1 | 0 |
| h | 0 | 1 | 1 | 0 | 1 |
| i | 1 | 0 | 1 | 1 | 0 |
| j | 0 | 1 | 0 | 1 | 1 |

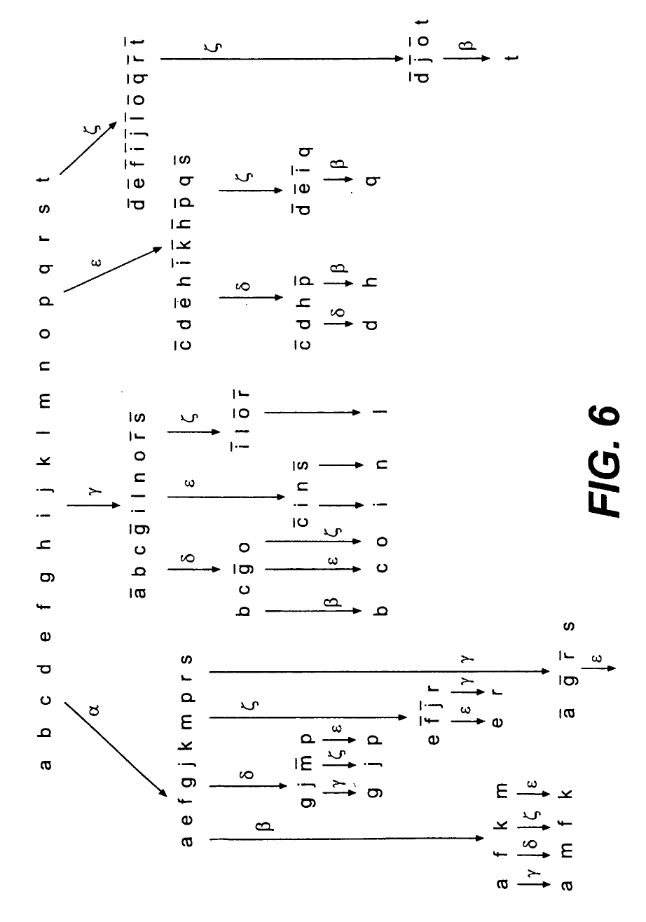

It is straightforward to implement for the separation of 20 proteins is shown in FIG. 6 (and the binding pattern is shown in Table 5). Again, we illustrate using an example in which each of the twenty proteins is capable of binding (not strongly) to three slinkers. For this separation, six slinkers will suffice. The protein left behind after exposure to a slinker which binds to it again is indicated by an overbar. For those proteins which bind strongly to their slinkers, the corresponding overbar is to be eliminated. For example, if g binds strongly to α, then there is no such thing as \overline{g} since α removed it all, leaving none for γ to bind.

TABLE 5
Binding Pattern for Twenty Proteins that each Bind to Three Slinkers

| | Slinkers | | | | | |
|---|---|---|---|---|---|---|
| Proteins | α | β | γ | δ | ε | ξ |
| a | 1 | 1 | 1 | 0 | 0 | 0 |
| b | 0 | 1 | 1 | 1 | 0 | 0 |
| c | 0 | 0 | 1 | 1 | 1 | 0 |
| d | 0 | 0 | 0 | 1 | 1 | 1 |
| e | 1 | 0 | 0 | 0 | 1 | 1 |
| f | 1 | 1 | 0 | 0 | 0 | 1 |
| g | 1 | 0 | 1 | 1 | 0 | 0 |
| h | 0 | 1 | 0 | 1 | 1 | 0 |
| i | 0 | 0 | 1 | 0 | 1 | 1 |
| j | 1 | 0 | 0 | 1 | 0 | 1 |
| k | 1 | 1 | 0 | 0 | 1 | 0 |
| l | 0 | 1 | 1 | 0 | 0 | 1 |
| m | 1 | 1 | 0 | 1 | 0 | 0 |
| n | 0 | 1 | 1 | 0 | 1 | 0 |
| o | 0 | 0 | 1 | 1 | 0 | 1 |
| p | 1 | 0 | 0 | 1 | 1 | 0 |
| q | 0 | 1 | 0 | 0 | 1 | 1 |
| r | 1 | 0 | 1 | 0 | 0 | 1 |
| s | 1 | 0 | 1 | 0 | 1 | 0 |
| t | 0 | 1 | 0 | 1 | 0 | 1 |

In a preferred embodiment of the present invention, the slinkers of the present invention are immobilized on a substrate. Possible substrates for immobilization include but are not limited to magnetic beads and wires, other types of beads, chromatographic columns (e.g. affinity or ion-exchange type), generally planar surfaces such as microslides, multiwell plates, chips, rods, tubes, fibers, optical fibers, and the like. The slinkers may be immobilized, for example, as microarrays on a chip, in parallel arrays or lanes on a glass microslide, or as “spots” on a glass microslide.

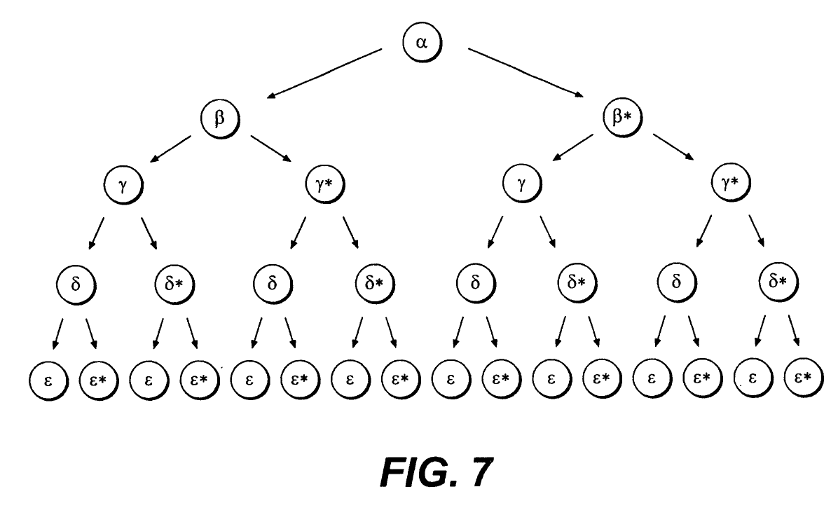

In a preferred embodiment of the present invention, microarrays coated with slinker spots or “wells” are used to apply RCP. Such an array is illustrated in FIG. 7, where the circles represent the spots or “wells” of a microarray and the characters α, β, γ, δ and ε represent five different slinkers that have been immobilized on the spots. First, an aliquot of the protein mixture to be analyzed is placed on the α-slinker coated spot and allowed to incubate. The supernatant (containing proteins that did not bind to the α-slinker) is then removed, leaving behind proteins which are bound to the α-slinker. The supernatant is placed on the β-coated spot on the left and labeled “β”. Proteins that became attached to the α-slinker spot are detached from the α-slinker spot by suitable means such as changing the ionic strength, pH, etc. They are then mixed with buffer and placed on the second β-coated spot, located at the right and labeled β*. (Note that slinker spots which receive protein that are bound to and subsequently detached from a previous slinker are labeled with (*) throughout FIG. 7, while those receiving supernatant are labeled only with the Greek character corresponding to the slinker.) The procedure is repeated using all slinkers in this array, in this case the five slinkers α, β, γ, δ and ε. If all proteins in the mixture have previously been labeled (for example, with a generic fluorescent label) the presence of labeled proteins in the last row of the array can be detected and the identity of the proteins established by correlation with known previously binding patterns.

Those of skill in the art will recognize that many techniques exist which are appropriate for immobilizing a slinker on a substrate. The immobilized slinkers may be covalently or physically linked to the substrate by techniques such as covalent bonding via an amide or ester linkage or by absorption, depending on the nature of the slinker. Further, the slinkers may be directly immobilized on the substrate, or they may be immobilized via “linker” or “spacer” molecules which serve to increase the distance between the slinker and the substrate. The use of spacer molecules may be advantageous in promoting slinker accessibility and in decreasing steric hindrance during slinker-protein interactions. Those of skill in the art will recognize that many suitable spacer molecules exist, including but not limited to secondary antibodies, peptides, alkyl chains, nucleic acid molecules, and the like. Any spacer molecule which serves to increase the distance between the slinker and the substrate without interfering with the interaction of the slinker and target molecule (e.g. a protein) may be used in the practice of the present invention.

The RCP methods of the present invention may be used as “stand alone” techniques, i.e. they may be used alone to effect the separation and classification of the proteins in a sample. However, those of skill in the art will recognize that RCP may be utilized in conjunction with other traditional separation techniques as well. Other techniques may be incorporated either prior to, during, or after the use of a set of slinkers. For example, prior to the use of RCP, cell extracts may be subjected to separation methods such as chromatography or electrophoresis in order to obtain a particular pool of proteins for analysis by RCP. Likewise, any subset pool of proteins generated during the RCP method may be further analyzed by other analytical techniques after completion of RCP or in the midst of utilizing a set of slinkers. Examples of such techniques include but are not limited to High Performance Liquid Chromatography (HPLC, both reverse and non-reverse phase), Fast Protein Liquid Chromatography (FPLC), ion exchange chromatography, affinity chromatography, sizing column chromatography, denaturing and non-denaturing polyacrylamide gel electrophoresis, capillary electrophoresis, ammonium sulfate precipitation, and the like.

Moreover, the RCP method can be used at different solution conditions, including pH, salt content, and ionic strength. These solution conditions can modulate the affinity of the slinkers for their ligands.

To facilitate detection of the molecules which are separated by the method of the present invention, fluorescence labeling of the molecules (e.g. proteins) contained in a protein mixture can be used. For example, the extract to be analyzed may be mixed with a generic fluorescent dye such as fluorescein, or with several more specific dyes for greater selectivity. In this manner, proteins which are separated by the practice of the present invention may be readily detected by well-established methods which are well-known to those of skill in the art.

In order to analyze a subset pool of molecules (e.g. proteins) which have bound to a slinker, the proteins typically are released from that slinker prior to exposure to the next slinker. This can be accomplished in a variety of ways, for example by changes in ionic strength or pH. However, in some cases, it may not be necessary to release the bound proteins from the slinker prior to exposure of the sample to the next slinker. This is especially desirable when the repeated use of some of the agents that detach magnetic beads from proteins runs the risk of permanent denaturation or other undesirable interactions with certain proteins. There are two limitations to this multiple bead-slinker attachment procedure. It is necessary that the binding site for the next intended slinker be sufficiently distant from the first binding site that its binding affinity is not significantly affected by the continued presence of the attached slinker at the first site; and, the beads used must have a sufficiently narrow size distribution that one can separate all proteins which have 2 beads attached from those that have only one bead attached.

In addition, RCP provides a ready means of classifying the proteins which are separated in this manner. Each separated protein will possess a distinct “binding signature” based on:

1) the protein's affinity to a subclass of slinkers, and

2) the protein's lack of affinity for other slinkers.

By “binding signature” we mean a summary of the binding affinities of a protein (or group of proteins) to a series of slinkers. For example, if an the series of slinkers consists of the four slinkers α, β, γ, and δ, and a protein binds to slinkers α and γ but does not bind to β and δ, then its binding signature can be given as: α+, β−, γ+, δ−.

Such binding signatures may be established for proteins (or other macromolecules e.g nucleic acids) for any of a variety of reasons, for example, to assess the pattern of differential protein expression during various biological states, such as disease states, pregnancy, changes related to aging, changes related to the administration of drugs (e.g. during drug screening and drug development), and the like. RCP may be utilized to assess the effect on patterns of protein expression of pollutants, diet or exercise, gene therapy, and the like. Those of skill in the art will recognize that many situations exist in which it would be of benefit to assess changes in the pattern of molecular expression, and that RCP may be utilized in any such suitable situation.

In addition, the binding signatures of individual proteins can be used to establish novel databases for classifying proteins according to their ability (or lack thereof) to bind a given set of slinkers. Such a database would allow the identification of the proteins or subsets of proteins contained in an unknown sample that is subjected to RCP. Individual proteins can be classified according to their binding signatures as described above, i.e. α+, β−, γ+, δ−.

It is expected that previously unknown proteins will be discovered by the use of RCP. Their binding signature will provide valuable structural and functional information since they will have to have certain 3-dimensional structures for the bindings which were found to have occurred, and also could not have the structures for the non-bindings. Further, by analogy to other proteins with their binding structures, functionality is suggested (but not proven).

Further, pathological signatures which are established based on RCP can be compiled into novel databases which include the entire pattern of protein expression for any suitable condition. A typical pathological signature may be comprised of the binding signatures of those proteins whose expression is changed by a particular disease state, or by exposure to a chemical (e.g. a drug) or for any reason of interest. The database thus produced can be used for the comparative analysis of unknown vs known samples in research or clinical applications in order to ascertain how patterns of protein expression change in response to a variable of interest (e.g. a disease, or administration of a drug, etc.). Such databases may be utilized in the diagnosis of diseases, or for drug screening and development, and the like.

In yet another embodiment of the present invention, the amount of protein which is bound to a slinker at a given step in the RCP process is modulated by controlling such binding reaction conditions as pH, temperature, ionic strength, counterion concentration, buffer type, additives (e.g. chelating agents or metal ions) and the like. In this embodiment, the protein sample is exposed to the slinker under defined conditions of, for example, ionic strength. Any proteins which bind to the slinker under those conditions are removed from the mixture. The condition of interest (e.g. ionic strength) is then adjusted and any proteins which bind to the slinker under the altered conditions are removed, and so on. (Alternatively, the condition of interest may be adjusted after the protein mixture is exposed to the slinker.) This process of adjusting a reaction or solution condition, and then removing the proteins that bind after each adjustment is continued until a desired level of separation of proteins is achieved. Further, this method of separation via adjustment of a reaction condition may be coupled with other means of protein separation, e.g. with the use of several different slinkers, or by combining the use of the adjustment of several different reaction conditions, e.g. adjustment of ionic strength and pH, either simultaneously or sequentially.

Proteins separated by the RCP method can subsequently be quantitated. This can be carried out by any of a variety of well-established methods which are known to those of skill in the art. For example, the proteins may be released from the immobilized slinker and quantitated spectroscopically. In some cases it may not be necessary to release the proteins from the immobilized substrate. For example, if the proteins have been previously labeled (e.g. with fluorescence) then fluorescence detection may be carried out directly while the protein is still attached to the slinker. In a preferred embodiment of the present invention, the slinker is a general-motif antibody that is immobilized on a magnetic bead. Protein quantitation is carried out (without releasing the protein) by monitoring the migration of the magnetic bead in a magnetic field. This embodiment of the present invention is described in detail below.

Quantification of protein expression in a sample can be obtained only as long as binding sites of the immobilized slinkers remain unsaturated. Proteins expressed in large quantities may saturate the binding sites, while those which are expressed at low concentrations will not saturate the binding sites. When appropriate, to detect the phenomenon of binding site saturation, the protein sample is simply diluted with an appropriate buffer. The sample is diluted and retested until an increase in protein binding is detected, which is the point at which less than complete saturation occurs. An additional dilution step is advisable to insure that a sufficient number of unsaturated sites remain available. Alternatively, the concentration of immobilized slinkers may be increased until the point of saturation is exceeded.

Use of Slinkers to Carry Out RCP

RCP is carried out by using slinkers as binding agents. Slinkers are substances (most often proteins or protein-polysaccharide combinations) which contain a functional domain that binds a selected fraction or subset of the different proteins contained in a protein mixture. A functional domain of a slinker binds a common motif in the subset of proteins. The proteins in the subset may or may not be otherwise related. The use of slinkers is therefore fundamentally different from the use of monoclonal and polyclonal antibodies, the latter of which routinely are used in biological applications to detect (e.g. in immunocytochemistry) or to separate (e.g. in affinity chromatography) specific proteins contained in cell extracts or mixtures. In those applications, specific antibodies are used since they bind only to one protein or at most to the different isoforms of the same protein

For example, the separation of proteins from a mixture via conventional affinity chromatography, requires the use of one specific antibody for each protein which is to be isolated. If several hundred proteins are present in a sample, and if it is desired to separate many or all of them, then an equal number of (i.e. several hundred) different specific antibodies would have to be used. This would be both time-consuming and costly. In contrast, RCP using slinkers offers a fundamental advantage over conventional procedures in that a much smaller number of slinkers may suffice to carry out the desired separation.

As pointed out before, slinkers can be small molecules or existing proteins (e.g. actin tubulin, etc.). In one embodiment of the instant invention, the slinkers which are utilized are “general-motif antibodies” which bind to two or more proteins. One way of creating general-motif antibodies is by first identifying amino acid sequences to use as antigens from which general-motif antibodies may be produced. This may be accomplished by comparing the amino acid sequences of those proteins which have been identified as belonging to, for example, a superfamily of proteins and identifying regions of homology among the proteins. Those of skill in the art will recognize that the amino acid sequences of such superfamilies are readily available from such sources as Gene Bank and Swiss Prot. Appropriate regions may contain identical amino acid sequences, or the amino acid sequences may be highly homologous, containing conservative substitutions. In a preferred embodiment, the homologous region comprises all or a portion of the protein which is generally accepted to be that which defines membership of the protein in the superfamily. For example, the sequence YLLSSGINGSFL (SEQ ID NO: 1) is shared by all 78 members of the Src homology 2 (SH2) superfamily of proteins. This sequence is located within the SH2 domain (i.e. within the defining domain) of this superfamily of proteins and is identical in all protein superfamily members identified to date. In contrast, other parts of the SH2 domain display only 48% identity from member to member.

Amino acid sequences identified in this manner may be produced synthetically or obtained by partial proteolysis of a purified protein according to methods which are well-known to those of skill in the art. This peptide sequence then serves as an antigen for generation of a purified, general-motif antibody, according to well-established methods which are well-known to those of skill in the art. When desired, the antibody may be conjugated to a dye molecule, thus making detection simple and reliable. Numerous suitable dye molecules and their method of use are well-known to those of skill in the art. This procedure allows the identification of many potential general-motif antibodies which then can be screened for efficacy in the RCP procedure.

Those of skill in the art will recognize that the design of general-motif antibodies also can be based on the secondary and tertiary structure of protein motifs and folds. Traditionally, proteins are classified not only by their shared primary sequence homology/identity, but also on the motifs and folds shared among parts (e.g. functional domains) of proteins. As the knowledge of high-resolution protein structures grows, the possibility of engineering general-motif antibodies based on structure also will grow.

General-motif antibodies may be generated against any protein motif which is antigenic, so long as the general-motif antibodies are useful in the practice of the present invention. Such motifs include primary structural elements (the primary sequence of amino acids), secondary structural elements (areas of local protein folding e.g. helices, bends, loops, etc.), and tertiary structural elements (areas where distal portions of the molecules interact, e.g. disulphide bridges, anionic bonding between oppositely charged side chains, etc). Such motifs may also include binding regions for molecules characteristically associated with a protein, or the bound molecules themselves (e.g. a coenzyme or cofactor). Such motifs include but are not limited to: homologous amino acid sequences such as those described above, DNA or RNA binding regions, co-factor binding regions or the cofactors themselves, sulfhydryl functions, saccharide binding regions or the saccharides themselves, sites of phosphorylation, and the like. General-motif antibodies may be generated against any antigenic motif which is characteristic of or common to a subset of proteins in a mixture.

In one embodiment of the present invention, a slinker may bind a large fraction (e.g. about half) of the proteins in the sample to which it is being exposed. However, those of skill in the art will recognize that slinkers which bind smaller or larger fractions of the proteins in a sample may also be suitable in the practice of the present invention. Any substance which binds a fraction of the proteins in the sample of interest may be used as a slinker in the practice of the present invention, so long as the set of slinkers into which that slinker has been assigned is capable of effecting an acceptable separation of proteins.

Earlier examples were provided in which each protein bound to various numbers of slinkers. In those examples, the proteins each bound to only two or to only three of the slinkers. The use of illustrative examples in which the number is constant (e.g. 2 or 3) is not a necessary condition. RCP schemes can be produced with the various proteins binding to different numbers of slinkers. In fact, in a particular mixture, some proteins may bind to only one slinker.

In a preferred embodiment of the present invention, the use of slinkers may be coupled with the use of other binding agents such as specific antibodies. For example, once a subset pool has been separated from a protein mixture via a slinker, specific antibodies may be utilized to identify specific proteins within the subset pool, or to remove specific proteins from the subset pool. Subsequently, the remaining proteins in the subset pool may again be subjected to RCP with additional slinkers. Other binding agents such as specific antibodies may be utilized at any step of an RCP procedure if so desired.

In short, a set of slinkers is two or more slinkers that can separate different constituents of interest in a sample containing a mixed population of constituents (e.g. proteins). Preferably, the set will include two or more slinkers, and may include 5 or 10 or more slinkers. In addition, the set also may include at least one, and possibly two or more specific antibodies.

In one embodiment of the present invention, slinkers may be used to separate non-interacting proteins as follows: Magnetic beads are coated with a slinker and are incubated with the sample of interest (e.g. a cell extract) in a suitable container, e.g. a single well of a multi-well plate. A set of proteins (optimally roughly half of those present in the sample) bind to the slinker on the beads and can be removed from the sample by removal of the beads via a magnetic field gradient. The slinker-bound proteins are released from the beads into, for example, a second well of the multi-well plate. This first separation step thus results in two pools of protein: one which binds to the first slinker and is removed with the magnetic beads, and one which does not, the latter pool being “left behind” when the magnetic beads are removed. Both protein pools are then incubated with a second slinker which is also bound to magnetic beads. Again, roughly half the proteins of each pool bind to the second slinker and are removed from the pool upon removal of the magnetic beads via a magnetic gradient. The proteins again are released from the magnetic beads. Each of the two protein pools thus is divided into two additional pools, resulting in a total of four protein pools, one of which binds both the first and second slinker, one of which binds the first but not the second slinker, one of which binds the second but not the first slinker, and one of which binds neither the first nor second slinker. This procedure may be repeated (e.g. with a third slinker in a set) as many times as is desired. Each subsequent exposure to a different slinker splits each protein pool which is assayed into two pools (one of which contains proteins which bind the slinker and one of which does not) until the desired level of separation is attained, e.g. until all proteins in the sample have been separated into pools each of which contains only a subset of related proteins, or, if desired, a single protein. Each protein subset or individual protein can be assigned a unique “binding signature” that indicates which slinkers it binds to, and which slinkers it does not bind to. The binding signature may yield enough information to identify the protein when the nature of the binding epitopes is known. For example, a protein which is bound by a general-motif antibody which was raised to the sequence YLLSSGINGSFL (SEQ ID NO: 1) most likely contains an SH2 domain. Confirmation of this identification may be made using specific monoclonal antibodies or by other means, such as an activity based assay.

In another embodiment, slinkers such as general-motif antibodies can be used in combination with specific antibodies to identify the proteins in a cell or tissue extract which interact with each other. For example, a mixture containing the protein a and three proteins b, c, and d which interact strongly with a may be analyzed in the following manner: the cell extract is mixed with magnetic beads coated with α, an anti-a antibody. The protein pairs a-b, a-c, and a-d are separated from the cell extract by magnetically removing the beads; all proteins that do not interact with a remain in the cell extract. The beads are then sequentially exposed to antibodies which are highly specific for b, c, and d, and which are themselves differentially labeled. When the beads are examined, it is possible to identify and quantify the concentrations of the a-b, a-c, and a-d pairs by detecting the differentially labeled specific antibodies. Similarly, the α antibody may be immobilized on a substrate such as a glass microslide or rod and non-bound proteins removed by rinsing the substrate prior to exposure to antibodies that are highly specific for b, c, and d.

Use of Magnetic Nanobeads to Quantitate the Amount of Protein in a Sample

The present invention provides a method and apparatus to quantitate the amount of protein in a sample once the protein is attached to a magnetic nanobead via a slinker. The method involves exposing a protein sample to a magnetic bead of relatively small diameter to which a slinker has been affixed. The number of molecules of slinker which are affixed to the bead is variable. For example, in some instances it may be advantageous to have only a single molecule of slinker attached. For other purposes, it may be advantageous to have a plurality of slinker molecules attached. Further, the molecules of slinker may all be the same (only one type of slinker) or different (more than one type of slinker e.g. two or more different types of antibodies per bead.) After an appropriate period of incubation, the beads are removed from the sample and the quantity of protein that is bound to the bead via the slinker is determined by measuring the speed of migration of the bead in a magnetic field in comparison to the speed of migration of a control bead or to a known speed stored in a lookup table or a known speed otherwise defined. Those of skill in the art will recognize that, since speed is distance traveled in a known amount of time, one can use either speed, or distance as the measurable.

In some embodiments of the present invention, the beads are exposed to a sample which is obtained, for example, by lysis of a cell. In another embodiment of the present invention, the beads are exposed to a sample by introducing the beads into a cell prior to lysis. Those of skill in the art will recognize that particles of a size similar to the beads of the present invention may be introduced into cells by a variety of established techniques. The macromolecules of interest (e.g. proteins, mRNA, or DNA) bind to the slinkers on the beads within the cell, and the cells may then be lysed by any of a variety of techniques which are well-known to those of skill in the art.

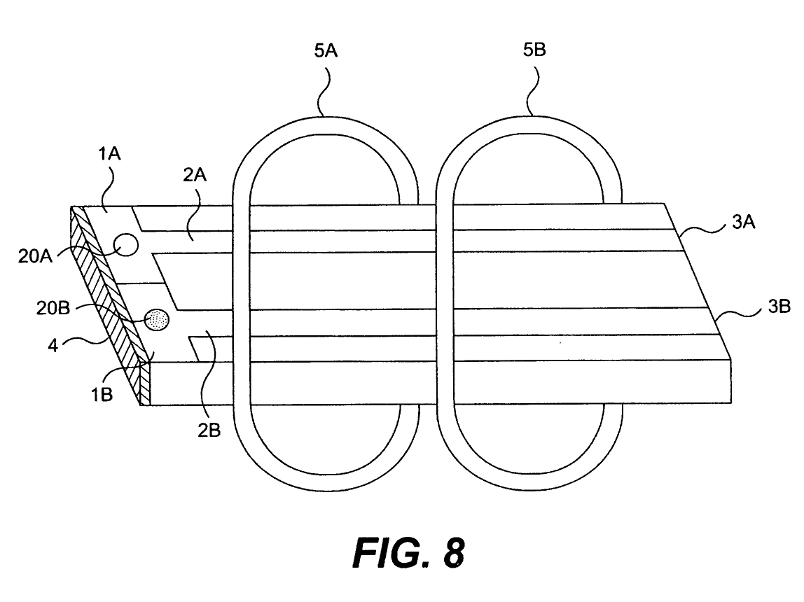

An apparatus for carrying out the measurement is illustrated in FIG. 8 and comprises a substrate containing a plurality of microchannels with adjacent loading areas at the proximal end of the channels. The cross-section of the microchannels is larger than the diameter of the beads. For simplicity, FIG. 8 depicts only two loading areas (1A and 1B) at the proximal ends of two channels, 2A and 2B. The channels are symmetrical about a longitudinal axis of the substrate. The exact dimensions of the channels and loading areas is not a crucial feature of the apparatus and may vary according to several factors, including the size of the beads being analyzed. The apparatus further comprises a plurality of electromagnets 4, 5A and 5B.

Those of skill in the art will recognize that a number of substrates would be appropriate for containing the microchannels of the device of the present invention. Examples of such substrates include but are not limited to: SiO₂, Si, Al₂O₃, GaAs, TiO₂, etc. Likewise, the exact form of the substrate is not crucial to the practice of the invention. For example, the channels may be fabricated on the horizontal surface of a glass microslide, or within a cylindrical-shaped substrate, and the like. Any substrate/channel design in which the beads may be deposited in a loading area and are free to migrate down a channel in response to a magnetic field gradient at a detectable rate, may be used in the practice of the present invention.

The slinker used in the method may be, for example, an antibody (either a general-motif antibody or a specific antibody). The slinker-coated beads are incubated with a specimen of interest, typically extracts from tissues and/or cell cultures. During the incubation, a protein or set of proteins attaches to the slinker on the beads, forming beads coated with slinker-protein complex (complexed beads, 20B). The complexed beads are removed from the sample and loaded onto a loading area 1B of a channel 2B. Identical control beads 20A (i.e. beads to which antibody has been attached and otherwise treated identically to the experimental beads, but not exposed to the protein sample) are loaded onto a loading area 1A of adjacent channel 2A and serve as the control. Initially, a first electromagnet 4 is on and applies a uniform and constant force on the beads which forces all beads to remain at loading areas 1A and 1B. Electromagnets (e.g. coaxial magnetic coils) 5A and 5B are turned on, creating a magnetic field gradient, and electromagnet 4 is turned off. The magnetic field-gradient coils produce a force which causes movement of the beads 20A and 20B towards the distal ends (3A and 3B) of the channels.

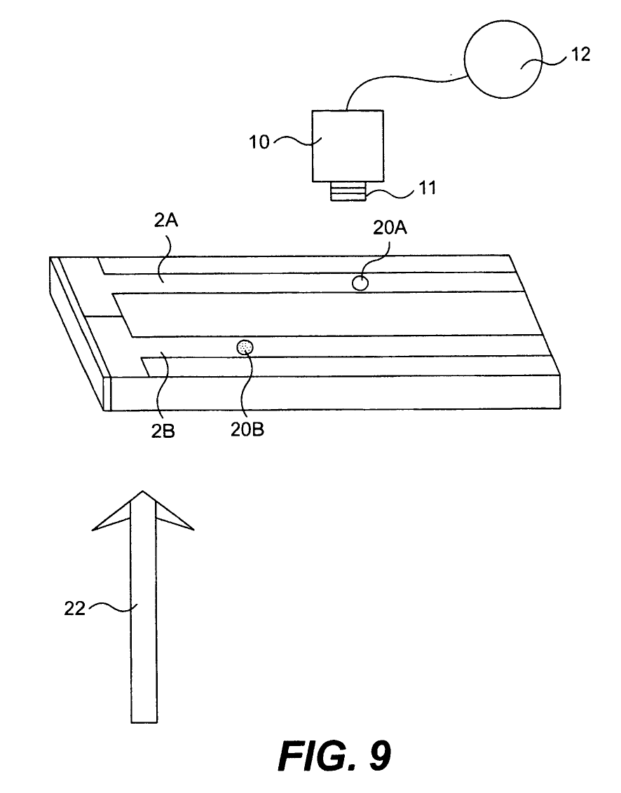

The magnetic force-induced displacement of the beads as a function of time is then measured by means such as using time-lapse video microscopy as illustrated in FIG. 5. In FIG. 9, a control bead 20A is depicted as migrating in a channel 2A ahead of complexed bead 20B, which is migrating in parallel channel 2B. The migration of both beads is tracked by a camera 10 with a lens 11, appropriately mounted over channels 2A and 2B in order to record images of the migration of beads 20A and 20B. The camera 10 is further connected to a computer 12 for analysis of the acquired images. Images of beads traveling in the channels may be acquired using, for example, either a digital camera or a silicon-intensifier target (SIT) camera. While FIG. 9 illustrates a control bead 20A run simultaneously with a complexed bead 20B, it is possible to compare results for the complexed bead 20B against stored empirical results, against predicted results, or against some other measure.

In one embodiment of the present invention, the movement of magnetic nanobeads within microchannels further may be differentiated by exposing the beads to a transverse field illustrated by arrow 22 during or prior to subjecting the beads to the final magnetic field. The transverse field may be, for example, a flow field, an electric field, or a preliminary magnetic field. The transverse field can be used to enhance the separation of controls from complexed beads to affect the length of movement in the channel, and for other reasons.

In one embodiment of the present invention, the movement of a single bead within a channel is monitored. The position of a bead can be defined by the centroid of the light intensity distribution or by the thresholded image of the bead using methods which are well-known to those of skill in the art. Both approaches offer a spatial resolution which is better than one pixel. For the thresholded image, the grey-scaled intensity image of the bead is “binarized”, i.e. the grey-scale image is analyzed pixel by pixel; pixel intensities smaller than a threshold value, typically chosen by the user, are assigned white; intensities larger than the same threshold are assigned black. The grey-scale image is thus transformed into a black and white image. The spatial resolution that already has been obtained using video-enhanced light microscopy is about 3 nm (Apgar J., Tseng, Y., Federov, E., Herwig, M. B., Almo, S. C. and Wirtz, D. (2000) Biophysical Journal, 79, 1095-1106).

In another embodiment of the present invention, the movement of several beads is tracked. In this case, the number of beads loaded into a channel must be small enough so that all of the beads in each channel can be tracked individually. From this data one can compute the position of, for example, the center of mass of the group of beads as a function of time. When displacement of the center of mass of the control beads is sufficiently different than that of the complexed beads, protein attachment can be detected. This approach is more precise than single bead tracking since it computes an average speed of migration from an ensemble of particles and thus eliminates much of the uncertainty due to differences in bead size.

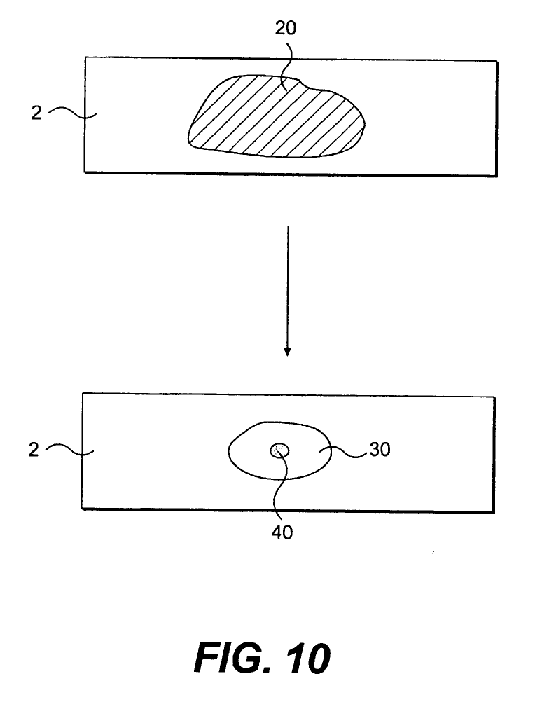

At particularly high field gradients, the speed of the beads may be fast enough to preclude frame-to-frame detection. In this case, an alternative embodiment of the present invention, “particle-packet centroid tracking” may be employed. In particle-packet centroid tracking, instead of tracking individual beads, the entire group of beads in a channel is analyzed as if it were a single object, or “packet”. The process is illustrated in FIG. 10. The grey-scaled intensity image 20 of a bead packet migrating in a channel 2 is “binarized”. The grey-scale image 20 is analyzed pixel by pixel; pixel intensities smaller than a threshold value, typically chosen by the user, are assigned white; intensities larger than the same threshold are assigned black. The grey-scale image 20 is thus transformed into a sharply delineated black and white image 30. The displacement of the packet's centroid 40 (i.e. center of mass position) is tracked.

Determination of the position of the centroid can be based either on light intensity measurements or on the threshold shape of the packet. Comparison of the migration of the centroids of control vs. complexed bead packets allow detection of protein binding to complexed beads because the complexed beads migrate more slowly due to their increased hydrodynamic drag, i.e. if the centroid of a complexed bead packet moves faster than its control, then protein is attached to the complexed beads. One advantage of particle-packet centroid tracking is that it automatically integrates size polydispersity by tracking many beads at the same time. Moreover, subpixel spatial resolution can be obtained.

In a preferred embodiment, images of beads traveling in adjacent channels are collected using either a digital camera or a SIT camera. These images are obtained with either bright field microscopy or fluorescence microscopy (for beads which have fluorescent tags attached, either directly to the bead or to the antibody). Distances traveled by the beads are computed by monitoring the center-of-mass displacements of the controls and, in the adjacent channel, of the beads that were incubated with the cell extract.

In most embodiments of tracking, the displacement of control beads and complexed beads are compared. The speed of movement of the beads will depend on the size of the bead, and when the beads are of an appropriate size, on the type and quantity of protein molecules that are attached to the beads. Further, measurements of complexed beads with known quantities of bound proteins whose size is known may also be used to generate standard calibration curves for use in quantitating the amount of an unknown protein which is bound to a magnetic bead.

One parameter that may be calculated is the spatial resolution between controls and complexed beads. By “spatial resolution” we mean the minimum measurable distance between individual beads or bead packets traveling in two different channels. This spatial resolution depends on:

1) the length of time used for image collection;

2) the strength of the forces applied to the beads (which will be proportional to the magnetic susceptibility of the beads to the magnetic field gradient, and to the volume of the beads);

3) the size of the proteins that attach to the bead during its incubation in a protein mixture; and

4) the number of proteins attached to the bead (which also depends on the number of antibodies present on the bead).

Calculation of the spatial resolution between control and complexed beads is carried out as follows for a magnetic bead of magnetic susceptibility μ and volume V, subject to a constant magnetic field gradient dH/dx and moving in a buffer of viscosity η. The magnetic susceptibility can be rendered constant in the superparamagnetic beads employed in the present invention by applying a constant magnetic field of ˜200 Gauss, which is readily obtained by magnets which are well known to those of skill in the art. The velocity of the bead is obtained from the balance of magnetic force F_{M} and friction force F₇₂ which both act on the moving bead, F_{M}=F_{η}. The magnetic force is proportional to μVdH/dx, and the friction force to 6πa ην, where ν is the velocity of the bead and a is its hydrodynamic radius. Therefore, ν is proportional to (μVdH/dx/6πa η, i.e. it is proportional to the inverse of the hydrodynamic radius of the particle. The volume V of the magnetic material that constitutes the bead is constant regardless of protein attachment to the bead. However, the hydrodynamic radius increases upon protein attachment. If a protein becomes attached to the bead, the radius increases by ε to a+ε. ν is then proportional to (μVdH/dx/6π(a+ε) η Therefore, the velocity of the complexed bead is less than that of the equivalent control bead. Note that, for a given magnetic field gradient and suspending buffer, the distance traveled by the centroid of the bead depends only on its hydrodynamic radius.

Under standard conditions, the differences between the distances traveled by a complexed bead and its corresponding control is larger than the spatial resolution of current particle tracking instruments. For example, if a=20 nm, F_{M}=0.1 pN, and η=1 cP (i.e. the viscosity of water), and if attachment of a protein changes the radius to a+ε=25 nm (i.e. the attached protein increases the hydrodynamic radius by 5 nm) then the distance traveled by the control bead during 10 seconds of image collecting time is given by 10 s×0.1 pN/6πa η or ˜265 μm, while for a bead which has protein attached, the distance traveled by that bead is reduced to 10 s×0.01 pN/6π(a+ε)η, or ˜212 μm The difference between the two distances measured in this manner is 53 μm, which is readily detectable by light microscopy. If desired, the force applied to the beads may be smaller by, for example, a factor of 100. Because this results in a smaller magnetic susceptibility, then one would simply apply the smaller force for a longer period of time (e.g. 100×10 seconds=1000 seconds) in order to achieve equivalent results i.e. the same difference in distance between the complexed and control beads. Collecting images for 1000 s instead of 10 s represents no technical difficulty as it only requires the user to video-tape the displacements of the particles for a longer time. These distances (e.g. 53 μm as calculated above) are much larger that the spatial resolution currently achievable using light microscopy. Therefore, protein attachment can readily be detected using the bead-position tracking approach of the present invention.

Further calculations illustrate the advantage of utilizing beads of a relatively small diameter such as those employed in the practice of the present invention. The relative distance of travel of a bead normalized to the bead size is simply [(α+ε)−α]/α=ε/α. The distance difference for a 1 μm bead and a 20 nm bead having a 5 nm protein attached would be:

For the 1 μm bead: (5/1000)×100%=0.5%

For the 20 nm bead: (5/20)×100%=25%

As can be seen, the relative distance is much larger for a small bead than for a large bead.

In a preferred embodiment of the present invention, the magnetic nanobeads to be utilized will have a radius in the range of about 5 to about 1000 nm. Magnetic beads which are currently commercially available fall outside this size range but may still be used in the practice of this invention. For example, Dynal (Norway) manufactures micron-size (2.8, 4.5, and 5.0 μm) spherical beads for protein-purification and cell-sorting applications. These beads are superparamagnetic, uniform in size, and contain a dispersion of magnetic material (Fe₂O₃ and Fe₃O₄). Beckman-Coulter also manufactures 1 μm-diameter superparamagnetic beads for cell-sorting applications. These beads also contain a dispersion of magnetic material.

The present invention provides magnetic nanobeads which fall within the preferred radius range of about 5 to about 50 nm. In a preferred embodiment of the present invention, the beads are hexagonal shaped crystals of γFe₂O₃ beads, which are made using the counterflow diffusion flame reactor in which iron carbonyl is utilized as the precursor. The counterflow diffusion flame reactor is described in U.S. Pat. No. 5,268,337 to Katz et al. and U.S. Pat. No. 5,650,130 to Katz et al. and the complete contents of both patents is herein incorporated by reference. Beads manufactured by this process are nearly all of exactly the same shape and have a very narrow size distribution. Further, the size can be extensively controlled. The small size of the magnetic beads of the present invention provides a distinct advantage over previously known magnetic beads for a number of reasons, for example: 1) Using small beads helps reduce the amount of undesirable contact between the bead surface and the attached protein, as compared to larger beads. Ideally, a protein bound to a bead does not contact the surface of the bead at all but makes contact only with appropriate atoms of the slinker. (The slinker itself is, of course, linked to the bead surface, either directly or indirectly via a spacer molecule.) Portions of the protein not directly involved with attachment to the slinker would remain free, tethered to the bead by the slinker but held away from the bead surface. This is advantageous because non-specific protein-bead surface contacts may cause denaturation of the protein. However, if a bead has a large radius (i.e. a small curvature) then a protein “perceives” the bead as being relatively flat, and the probability of contact between the protein and the bead surface is higher than if the bead's radius is small (i.e. if the bead's curvature is large). Using small beads therefore reduces the probability of contact between the bead surface and the attached protein, thus decreasing the possibility of protein denaturation. 2) The difference in speed of migration between a control bead and a complexed bead is much more readily detectable if the bead is small than if it is large when compared to the size of the protein. (See calculation above.) This is because the speed of migration of magnetic beads in a magnetic field gradient depends inversely on the hydrodynamic radius of the bead. This radius will be barely changed if, for example, a 5 nm protein is attached to a 1 μm radius bead. However, if the same protein is attached to a 20 nm radius bead, the relative increase in radius is much larger and readily detectable. This point is further illustrated in the calculations presented above.

3) The statistics on packet position is significantly improved for the same volume or field of view when smaller beads are used. This is because the number of beads in a given field of view will be much larger when using smaller beads than when using larger beads. 4) Smaller beads diffuse in solution more rapidly than larger beads, dramatically enhancing the rate of collision between the beads and proteins in solution. Thus, if a cell extract contains very few copies of a given protein, using small beads will increase the probability of contact and reduce the occurrence of false negatives in an assay.

While the preferred magnetic beads to be used in the practice of the present invention are those described above, those of skill in the art will recognize that any magnetic bead of any composition and any diameter, which allows the type and/or quantity of protein bound to it to be ascertained by the methods of the present invention, may be utilized in the practice of the present invention.

In one embodiment of the present invention, the relative levels of expression of proteins in different specimens may be obtained from the distances traveled by a bead in a fixed length of time. For example, two specimens (e.g. normal vs cancer cells) can be compared side-by-side. If proteins are expressed at different levels, the “pattern” of progression of the magnetic beads will be different; beads with more bound protein (i.e. from a sample which contains a higher amount of the protein) will travel a lesser distance than beads containing less bound protein. Further analysis of the bound proteins (e.g. amino acid analysis or sequencing) may also be carried out by sampling the beads at the ends of the channels.

This method applies to the detection of proteins which not only are over-expressed in a sample (or which exist in higher concentrations in a mixture), but also to proteins which are down-regulated (or exist in lower concentrations) compared to a control. For example, when using beads attached to a particular antibody α, the corresponding protein a may be present or expressed at lower levels in a diseased specimen than in a normal, control specimen. When using an excess of particles, in the case where protein a is down-regulated, a majority of α beads will travel a large distance when incubated with the disease sample than those that were incubated in the normal specimen due to the lower amount of attached protein.

In another embodiment of the present invention, magnetic beads of different sizes may be utilized in order to enhance protein separation. This may be especially useful for proteins which are interactive. Consider two sets of beads; the range of sizes of the beads in one set is relatively uniform and differs sufficiently from the range of sizes of the beads in the other set that there is a clear difference in sizes. For example: a protein mixture contains proteins a and b (plus ab complex) which bind antibodies α and β, respectively. Antibody α is immobilized on beads of a relatively large size. Antibody β is immobilized on beads of a relatively small size. The protein mixture is first exposed to antibody α, which will bind all of protein a. The protein mixture then is exposed to antibody β thus binding the protein b. When all the beads are placed in a magnetic field gradient, the large beads (the α beads) will move much more rapidly than the β beads (regardless of the quantities of protein bound), because of the large difference in bead size.

To improve the measurement of protein concentrations in a given mixture, it is sometimes desirable to utilize particles to which only a single molecule of slinker is attached. For example, the attachment of a protein to a particle (itself connected to only one slinker) will make a larger change in the overall hydrodynamic radius of the combination than if more proteins and, and therefore more slinkers, were attached. As a result, the particle can be detected more easily.

The attachment of a single (as opposed to more than one) slinker to a particle is greatly facilitated by using small (e.g., 5-50 nm-radius) magnetic particles. In a preferred embodiment, a very large excess of small magnetic particles is incubated with a relatively small concentration of a given slinker. This greatly increases the probability that each particle becomes attached to either zero or one slinker, as opposed to more than one slinker. Using a magnetic field gradient, all particles are subject to a force; those particles which became attached to one slinker during the precedent incubation will move more slowly than the particles attached to no slinker. As discussed above, this is because the attachment of an slinker to a small particle increases greatly the overall hydrodynamic radius of the particle when that particle is small, much more so than when that particle is large compared to the slinker. In contrast, the particles that are not attached to a slinker molecule will move rapidly, and (as a result) can be separated from the particles that are attached to a slinker molecule. This separation thus yields particles bound to a single slinker, which can then be used for protein detection, e.g. via the RCP. Note that the unattached particles can be reused for attachment to more of the same or to a different slinker.

In another embodiment of the present invention, additional slinkers are positioned at or near the distal ends of the channels through which the beads migrate. One purpose for these additional slinkers is to “capture” the beads or a subset of the beads as they migrate through the channel. For example, such “capturing slinkers” may be positioned (e.g. via immobilization) at or near the distal end of a channel. They may be, for example, specific antibodies or general-motif antibodies directed to a motif on a protein of interest. The protein of interest is already attached to a migrating bead or beads via a slinker as described above. The bead-protein complex will migrate through the channel and be stopped upon contacting the capturing slinker because the capturing slinker will bind to the protein of interest, sequestering the protein and the attached bead. In this way, for example, beads with no bound protein, or beads bound to a protein which is not recognized by the capturing slinker, can be separated from beads bound to a protein which is recognized by the capturing slinker. Beads with no bound protein, or beads bound to a protein which is not recognized by the capturing slinker, would freely migrate past the capturing antibody.

Use of Optical Fibers to Detect Protein Binding to Immobilized Slinkers

In a further embodiment of the instant invention, binding events between immobilized slinkers and proteins may be detected using optical fibers. In a preferred embodiment, an array of optical fibers, each coated with a different slinker, is utilized. This array of fibers is dipped into the cell extract to be examined, to which a generic fluorescent dye has been added. The optical-fiber array is removed from the cell extract and a laser beam is shined into each optical fiber. If the fiber end is fluorescent, then a protein that interacts with the slinker on the fiber is present in the extract. This detects the presence of attached proteins (only those fibers with attached proteins will fluoresce) and measures the amounts of proteins that are attached to the fibers (the amount of fluorescence will be proportional to the amount of protein bound to the fiber). The method can be automated for rapid analysis of the proteins in a sample.

Kit

The present invention also provides a kit for the determination of the binding signatures of molecules (e.g. proteins) via RCP. Such a kit comprises a set of slinkers for use in determining the binding signature of one or more molecules correlated with a given condition of interest. The condition of interest may be a disease state. The slinkers may be antibodies (e.g. specific or general-motif antibodies) or other slinkers as described above. Further, the slinkers may be immobilized, for example on a substrate such as a magnetic bead. The kit may further comprise such items as buffers, instructions for use, and database materials (e.g. software) for carrying out comparative analyses of the results obtained with the slinkers.

While the invention has been described in terms of its preferred embodiments, those skilled in the art will recognize that the invention can be practiced with modification within the spirit and scope of the appended claims.

We claim:

1. A method for separating proteins that are present in a mixture of proteins, comprising,
exposing said mixture of proteins to a slinker, wherein said step of exposing creates a positive protein pool and a negative protein pool, said positive protein pool comprising proteins that bind to said slinker and said negative protein pool comprising proteins that do not bind to said slinker;
separating said positive protein pool from said negative protein pool; and
performing said steps of exposing and separating multiple times.

2. The method of claim 1 wherein each of said positive protein pool and said negative protein pool include a plurality of proteins.

3. The method of claim 1 wherein said slinker is selected from the group consisting of specific antibodies, substrates, substrate analogs, co-factors, coenzymes, inhibitors, protein-binding DNA and RNA sequences, metal ions, saccharides, spacer molecules with amino or sulfhydryl functions, actins, tubulins, integrins, selecting, cadherins, intermediate filaments, vimentin, neurofilaments, and keratins.

4. The method of claim 1 wherein said slinker is immobilized on a substrate.

5. The method of claim 4 wherein said substrate is selected from the group consisting of magnetic beads, wires, rods, fibers, microslides, and chromatographic columns.

6. The method of claim 4 wherein said slinker is immobilized on said substrate by attachment to a spacer molecule.

7. The method of claim 1 further comprising the step of adjusting a condition of said exposing step.

8. The method of claim 7 wherein said condition is selected from the group consisting of pH, temperature, ionic strength, and buffer composition.

9. The method of claim 1 further comprising the step of releasing said positive protein pool from said slinker.

10. The method of claim 1 further comprising a step selected from the group consisting of performing chromatography of said mixture of proteins and carrying out electrophoresis of said mixture of proteins.

11. The method of claim 1 wherein said proteins in said mixture of proteins are labeled with a detectable label.

12. The method of claim 11 wherein said detectable label is fluorescent.

13. The method of claim 1 wherein said slinker is labeled with a detectable label.

14. The method of claim 13 wherein said detectable label is fluorescent.

15. The method of claim 1 wherein selection of said slinker is made in conjunction with information obtained through genomic screening.

16. The method of claim 1 wherein said proteins are non-interactive.

17. The method of claim 1 wherein said proteins are interactive.

18. The method of claim 1 wherein said slinker is a general-motif antibody.

19. A method of producing a general-motif antibody, comprising,
identifying a motif which is common to all members in a family of proteins; and,
creating an antibody specific for said motif, said antibody thus formed being able to bind said members of said family of proteins.

20. The method of claim 19 wherein said motif is selected from the group consisting of a region of primary structural homology, a region of secondary structural homology and a region of tertiary structural homology.

21. A protein classification database, comprising
a plurality of binding signatures, wherein each binding signature is generated from Repeated Classification Procedure data and is unique for each protein in said protein classification database.

22. A method of generating a protein classification database for a plurality of proteins, comprising the steps of:
(a) exposing each of said plurality of proteins to a plurality of slinkers,
(b) detecting which of said plurality of slinkers bind to each of said proteins and which of said plurality of slinkers do not bind to each of said proteins,
(c) establishing a binding signature for each of said proteins based on the pattern of binding of said slinkers to each of said proteins, and
(d)compiling said binding signatures into a database.

23. The method of claim 22 wherein said plurality of slinkers comprises slinkers selected from the group consisting of specific antibodies, substrates, substrate analogs, co-factors, coenzymes, inhibitors, protein-binding DNA and RNA sequences, metal ions, saccharides, spacer molecules with amino or sulfhydryl functions, actins, tubulins, integrins, selectins, cadherins, intermediate filaments, vimentin, neurofilaments, and keratins.

24. The method of claim 22 wherein said plurality of slinkers comprises at least one general-motif antibody.

25. A method for determining the amount of a protein in a sample, comprising the steps of,
a) contacting said sample with a magnetic nanobead, wherein said magnetic nanobead is attached to at least one slinker which binds said protein, and wherein said step of contacting is carried out under conditions such that a quantity of said protein binds to said at least one slinker, forming a protein-slinker-magnetic nanobead complex;
b) measuring migration of said protein-slinker-magnetic nanobead complex in a magnetic field; and
c) determining an amount of protein bound to said magnetic nanobead based on the migration measured in said measuring step.

26. The method of claim 25 wherein said at least one slinker is an antibody.

27. The method of claim 26 wherein said antibody is a general-motif antibody.

28. The method of claim 26 wherein said antibody is a specific antibody.

29. The method of claim 25 further comprising the step of exposing said magnetic nanobead to a transverse field during said step of measuring.

30. The method of claim 29 wherein said transverse field is selected from the group consisting of an electric field, a flow field, and a magnetic field gradient.

31. The method of claim 25, further comprising the step of performing steps a-c with magnetic nanobeads of at least two different sizes, wherein a magnetic nanobead of one of said different sizes is coated with at least one different slinker than a magnetic nanobead of any other of said different sizes.

32. The method of claim 25 wherein said measuring step measures a distance traveled by said magnetic nanobead.

33. The method of claim 25 wherein said measuring step measures a speed of migration.

34. The method of claim 25 wherein said step of measuring is performed using a camera and image analysis equipment.

35. The method of claim 25 wherein said magnetic nanobead is attached to a single slinker molecule.

36. The method of claim 25 wherein said magnetic nanobead is attached to a plurality of slinker molecules.

37. An apparatus for measuring protein content in a sample, comprising:
a magnetic nanobead;
at least one slinker associated with said magnetic nanobead, said at least one slinker being capable of binding a protein of interest;
a means for monitoring migration of said magnetic nanobead in a magnetic field; and
a means for calculating protein content bound to said at least one slinker on said magnetic nanobead based on said migration identified by said means for monitoring.

38. The apparatus of claim 37 further comprising:
a substrate with at least one microchannel having a cross-section that is larger than a diameter of said magnetic nanobead; and
sensors which sense movement of said magnetic nanobead in said at least one microchannel.

39. The apparatus of claim 37 wherein said at least one slinker is an antibody.

40. The apparatus of claim 39 wherein said antibody is a general-motif antibody.

41. The apparatus of claim 39 wherein said antibody is a specific antibody.

42. The apparatus of claim 37 wherein said at least one slinker is associated to said magnetic nanobead by covalent binding.

43. The apparatus of claim 37 wherein said at least one slinker is associated to said magnetic nanobead by ionic binding.

44. The apparatus of claim 37 wherein said magnetic nanobead has a radius in the range of about 5 to about 1000 nm.

45. The apparatus of claim 37 wherein said apparatus further comprises a second slinker located at a distal end of said at least one microchannel.

46. A magnetic nanobead with at least one bound slinker, said magnetic nanobead having a radius in the range of about 5 to about 1000 nm.

47. The magnetic nanobead of claim 46 wherein said at least one bound slinker is a general-motif antibody.

48. The magnetic nanobead of claim 46 wherein said at least one bound slinker is bound to said magnetic nanobead covalently.

49. The magnetic nanobead of claim 46 wherein said at least one bound slinker is bound to said magnetic nanobead ionically.

50. A method for quantitating proteins which are present in a mixture of proteins, comprising the steps of,
a) labeling said proteins in said mixture of proteins with a fluorescent dye in order to generate a mixture of fluorescently labeled proteins,
b) exposing said mixture of fluorescently labeled proteins to an optical fiber array, wherein said optical fiber array is comprised of a plurality of optical fibers each of which is coated with a different slinker;
c) removing said optical fiber array from said mixture of fluorescently labeled proteins;
d) illuminating said optical fibers with a laser;
e) measuring the amount of fluorescence generated by each optical fiber in said illuminating step;
f) correlating said amount of fluorescence with a quantity of protein.

51. The method of claim 50 wherein said slinker is a general-motif antibody.

52. An array for identifying proteins in a mixed sample of proteins, comprising
a plurality of optical fibers each of which is coated with a different slinker.

53. The array of claim 52 further comprising means for quantitating said proteins.

54. The array of claim 53 wherein said means for quantitating is a fluorometer.

55. The array of claim 52 wherein at least one of said slinkers is a general-motif antibody.

56. An array for identifying proteins in a mixed sample of proteins, comprising
a plurality of different slinkers immobilized on a common substrate.

57. The array of claim 56 wherein each of said slinkers is present as a separate spot on said substrate.

58. The array of claim 56 wherein each of said slinkers is present in a separate well on said substrate.

59. The array of claim 56 wherein at least one of said slinkers is a general-motif antibody.

60. A method for assessing disease states in a patient, comprising the steps of:
identifying by Rapid Classification Procedure a signature of protein binding for proteins in a sample obtained from said patient, and
comparing said signature of protein binding to stored signatures indicative of disease states.

61. A kit for determining the binding signatures of proteins via Reverse Classification Procedure, comprising,
a set of slinkers.

62. The kit of claim 61 wherein at least one slinker of said set of slinkers is a general-motif antibody.

63. The kit of claim 61 wherein said set of slinkers is immobilized on a substrate.

64. The kit of claim 63 wherein said substrate is a magnetic bead.

65. The method of claim 22, wherein steps a and b are repeated multiple times.
